(19) United States
(12) Patent Application Publication
(10) Pub. No.: US 2007/0172431 A1
(43) Pub. Date: Jul. 26, 2007

(54) Methods, systems and compositions for skin care

(76) Inventor: Matthew Galumbeck, Virginia Beach, VA (US)

Correspondence Address: TROUTMAN SANDERS LLP, 600 PEACHTREE STREET , NE, ATLANTA, GA, 30308, US

(21) Appl. No.: 11/526,328

(22) Filed: Sep. 25, 2006

Related U.S. Application Data
(60) Provisional application No. 60/760,574, filed on Jan. 20, 2006.

(51) Int. Cl. A61K 8/97; A61K 36/886; A61K 36/899; A61K 36/48

(52) U.S. Cl. 424/47; 424/74; 424/59; 424/61; 424/744; 424/750; 424/757

(57) ABSTRACT

The present invention describes systems, methods, and compositions for treatment and maintenance of skin. The invention includes a skin care system that addresses the problems of skin aging and inflammation. The invention further includes skin care systems, methods and compositions containing active ingredients that may inhibit matrix metalloproteniases (MMPs) or that may inhibit inflammatory processes of cells, prevent moisture loss, improve skin tone (or firmness), stimulate the production of collagen and elastin, decrease the breakdown of collagen and elastin, and prevent damage to the skin caused by environmental factors.

### CROSS-REFERENCE TO RELATED APPLICATIONS

This patent application claims benefit of priority of provisional application U.S. Ser. No. 60/760,574, filed Jan. 20, 2006.

### TECHNICAL FIELD

The invention relates to methods, systems, and compositions for skin care comprising combinations of skin care actives. More specifically, the invention relates to methods, systems and compositions for regulating and improving the condition of skin.

### BACKGROUND

Human skin is a complex organ that extends over the entire body. There are different types of skin at different sites of the body; for example, facial skin is different from that of the scalp, and the skin on the palm of the hand is different from skin on the back of the hand. Although the type of skin can vary over a person's body, skin is generally composed of two main layers of tissue. The epidermis (or cuticle) is the outermost layer. The epidermis further includes three superficial and two deep layers. The dermis (also called the corium, cutis vera, or the true skin) lies below the epidermis and includes a papillary layer above and a reticular layer below.

Skin is important for regulating body temperature and for providing a barrier to infectious organisms. With respect to temperature regulation, sweat glands produce sweat that cools the skin as it evaporates. Regarding the barrier function, the epidermis includes a layer of overlapping, flat, dead cells organized in columnar clusters. These epidermal cells are sealed with tightly packed, multi-lamellar lipid sheets that are covalently attached to the cell membranes. The epidermis is thicker in areas like the palms and soles that withstand more daily wear and tear than other parts of the body. Moisture content of the skin is critical for maintaining its barrier function. Dry skin will crack and allow infectious organisms an opening into the bloodstream.

With respect to skin appearance, collagen and elastin are proteins that help maintain skin suppleness and elasticity. It is believed that collagen and elastin production in human skin cells decreases as humans age, and that the rate of collagen breakdown increases. It is thought that these processes result in the formation of fine lines and wrinkles in the skin.

Skin inflammation and aging are closely related phenomena. So similar are the processes involved with both, that aging is sometimes described dermatologically as a chronic low grade inflammatory condition. In acute inflammation, there is typically a respiratory burst of neutrophil activity that initiates one or more cascades that involve a change in the oxidation state of the cell. Acute inflammation is also characterized by mast cell degranulation where serotonin is produced, which acts as a signal transduction factor. Following that, excited oxygen species are generated, such as superoxide anion, and these damage the lipid-rich membranes and activate the chemical mediators of proinflammation and inflammation.

A variety of substances have been applied to the skin to improve its appearance, generally by affecting the epidermis or by treating the dermis. Some treatments have involved retinoic acid for the treatment of acne and for the treatment of skin keratoses, wrinkles, warts, psoriasis, eczema and dandruff. There are known side effects to using retinoids and such treatments must be monitored carefully.

What are needed are skin care systems that address the problems of skin aging and inflammation. What are also needed are skin care systems, methods and compositions that comprise active ingredients that may inhibit matrix metalloproteniases (MMPs) or that may inhibit inflammatory processes of cells, prevent moisture loss, improve tone (or firmness), stimulate the production of collagen or elastin, decrease the breakdown of collagen and elastin, or prevent damage to the skin caused by environmental factors.

### SUMMARY

The present invention comprises systems, methods and compositions for treatment and maintenance of skin. The invention includes compositions that may inhibit MMPs or that may inhibit inflammatory processes of cells, prevent moisture loss, improve tone (or firmness), stimulate the production of collagen or elastin, decrease the breakdown of collagen and elastin, or prevent damage to the skin caused by environmental factors. The present invention further includes methods for making the compositions. In addition, the present invention includes methods for using the compositions to treat and maintain skin.

### DETAILED DESCRIPTION

The present invention comprises systems, methods and compositions for treatment and maintenance of skin. The present invention comprises systems for treatment of skin comprising methods comprising administering compositions comprising bioactive compounds that are effective for inhibiting MMPs or for inflammatory processes of cells, preventing moisture loss, improving tone or firmness, stimulating the production of collagen or elastin, decreasing the breakdown of collagen or elastin, or preventing damage to skin caused by environmental factors.

Though not wishing to be bound by any particular theory, it is currently believed that inflammation may cause a multifactorial response that includes the release of elastase from leukocytes, a reduction in free radical scavengers, a decrease in collagen production, and an increased activity of MMPs. Elastase released from leukocytes may be a potent proteinase that breaks down collagen and elastin, leading to the loss of cellular moisture. Chronic inflammation may decrease intracellular oxygen free radical scavengers such as superoxide dismutase (SOD). Consequently, a rise in oxygen free radical molecules may create lipid peroxides that decrease the barrier properties of the skin and accelerate dermal aging. MMPs play a role in collagen degradation. MMPs may inhibit fibroblast skin cell proliferation, break down extracellular collagen, and inhibit the formation of new collagen.

In healthy individuals, several naturally occurring antioxidant defense systems detoxify the various reactive oxygen species (ROS) or free radicals and, thereby, aid in normal cell and tissue integrity and function. These systems of detoxification involve the stepwise conversion of ROS or free radicals to less toxic species by the concerted activities of certain antioxidative enzymes. These antioxidative enzymes are members of a larger class of molecules known as “oxygen radical scavengers” or “lazaroids” that have an ability to scavenge and detoxify ROS and free radicals. Vitamins A, C, E, and related antioxidant compounds, such as Scarotene and retinoids, are also members of this larger class. In healthy individuals, sufficient levels of antioxidative enzymes and other lazaroids are present both intracellularly and extracellularly to efficiently scavenge sufficient amounts of ROS and free radicals to avoid significant oxidative damage to cells and tissues.

Additionally, though not wishing to be bound by any particular theory, it is currently believed, skin aging is a process that includes the interaction of multiple intracellular pathways that adversely affect levels of collagen and elastin. Moisture content is also involved in proper skin function. Accordingly, addressing skin aging may include treatments for inflammation-associated skin damage and moisture content.

Skin is also affected by the oil glands present in the skin, whether by the number, the activity level, or the architecture of the glands or associated bacteria. Skin is often classified in one of three categories: normal, oily, or dry, based on the oil gland presence in the skin. This adds to the complexity of treating the skin.

Methods of the present invention comprise treating each type of skin, whether young or aging, or normal, oily, dry, or combinations thereof, by administering an effective amount of one or more compositions taught herein, or use of a system for treating skin disclosed herein. Compositions taught herein are effective for enhancing or improving barrier properties of skin and improving the appearance of skin, including preventing moisture loss, improving tone and firmness, stimulating the production of collagen and elastin, decreasing the breakdown of collagen and elastin, and preventing damage to the skin caused by environmental factors, including but not limited to, ultraviolet (UV) or other radiation, stress, and smoking.

The compositions of the present invention comprise active agents that are effective for enhancing or improving barrier properties of skin and improving the appearance of skin, including preventing moisture loss, improving tone and firmness, stimulating the production of collagen and elastin, decreasing the breakdown of collagen and elastin, and preventing damage to the skin caused by environmental factors, including but not limited to, UV or other radiation, stress, and smoking. The compositions of the present invention may also be anti-aging compositions because the compositions can be effective in reducing the appearance of fine lines and wrinkles in facial skin.

In one embodiment, the compositions of the skin care systems of the present invention comprise one or more skin care effective agents, referred to herein as bioactive modulators, in combination with a dermatologically acceptable vehicle, wherein the ratio between the mixture of one or more active ingredients and the dermatologically-acceptable vehicle varies from about 1:1000 to about 13:1, as calculated on a weight to weight basis (w/w). In another embodiment, the compositions of the skin care systems (i.e., bioactive modulators) in combination with a dermatologically acceptable vehicle exist in a ratio between the mixture of one or more active ingredients and the dermatologically-acceptable vehicle from about 1:850 to about 10:1, as calculated on a weight to weight basis (w/w). In yet another embodiment of the present invention, the compositions of the skin care systems in combination with a dermatologically acceptable vehicle exist in a ratio between the mixture of one or more active ingredients and the dermatologically-acceptable vehicle from about 1:675 to about 7.5:1, as calculated on a weight to weight basis (w/w). In yet another embodiment, the compositions of the skin care systems in combination with a dermatologically acceptable vehicle exist in a ratio between the mixture of one or more active ingredients and the dermatologically-acceptable vehicle from about 1:500 to about 5:1, as calculated on a weight to weight basis (w/w). In yet another embodiment, the compositions of the skin care systems in combination with a dermatologically acceptable vehicle exist in a ratio between the mixture of one or more active ingredients and the dermatologically-acceptable vehicle from about 1:200 to about 2.5:1, as calculated on a weight to weight basis (w/w). In yet another embodiment, the compositions of the skin care systems in combination with a dermatologically acceptable vehicle exist in a ratio between the mixture of one or more active ingredients and the dermatologically-acceptable vehicle from about 1:50 to about 1:1, as calculated on a weight to weight basis (w/w). This variation of ranges permits compositions to be tailored to the needs of individuals, based on the location of the skin to be treated, such as face, hands, arms, skin care type, and the nature of the exposure to various skin irritants such as sunlight, wind abrasion, radiation, or other insults.

The combinations of active ingredients and vehicle components work together to achieve improved results to treat skin with respect to decreasing the inflammatory response, improving moisture levels, increasing cellular antioxidant capabilities, increasing collagen production, and decreasing MMP activity. The active ingredients include, but are not limited to, Aloe Vera (comprising aloe barbadensis leaf juice, an anti-inflammatory), Vitamin E (tocopheryl acetate, an antioxidant), Colhibin (hydrolyzed rice protein, an MMP inhibitor), and Elhibin (glycine soya protein, a leukocyte esterase inhibitor).

By way of example, an effective ratio of aloe barbadensis leaf to dermatologically acceptable vehicle may range from about 1:1000 to about 13:1, as calculated on a weight to weight basis (w/w). Alternatively, an effective ratio of aloe barbadensis leaf to dermatologically acceptable vehicle may range from about 1:850 to about 10:1. As another alternative, an effective ratio of aloe barbadensis leaf to dermatologically acceptable vehicle may range from about 1:675 to about 7.5:1. As yet another alternative, an effective ratio of aloe barbadensis leaf to dermatologically acceptable vehicle may range from about 1:500 to about 5:1. As yet another alternative, an effective ratio of aloe barbadensis leaf to dermatologically acceptable vehicle may range from about 1:200 to about 2.5:1. As yet another alternative, an effective ratio of aloe barbadensis leaf to dermatologically acceptable vehicle may range from about 1:50 to about 1:1. All of the effective ratios are as calculated on a weight to weight basis (w/w).

By further way of example, an effective ratio of tocopheryl acetate to dermatologically acceptable vehicle may range from about 1:675 to about 1:500, as calculated on a weight to weight basis (w/w). Alternatively, an effective ratio of tocopheryl acetate to dermatologically acceptable vehicle may range from about 1:650 to about 1:525. As another alternative, an effective ratio of tocopheryl acetate to dermatologically acceptable vehicle may range from about 1:600 to about 1:550. All of the effective ratios are as calculated on a weight to weight basis (w/w).

By further way of example, an effective ratio of hydrolyzed rice protein to dermatologically acceptable vehicle may range from about 1:200 to about 1:50, as calculated on a weight to weight basis (w/w). Alternatively, an effective ratio of hydrolyzed rice protein to dermatologically acceptable vehicle may range from about 1:150 to about 1:75. As another alternative, an effective ratio of hydrolyzed rice protein to dermatologically acceptable vehicle may range from about 1:125 to about 1:100. All of the effective ratios are as calculated on a weight to weight basis (w/w).

By further way of example, an effective ratio of glycine soya protein to dermatologically acceptable vehicle may range from about 1:200 to about 1:50, as calculated on a weight to weight basis (w/w). Alternatively, an effective ratio of glycine soya protein to dermatologically acceptable vehicle may range from about 1:150 to about 1:75. As another alternative, an effective ratio of glycine soya protein to dermatologically acceptable vehicle may range from about 1:125 to about 1:100. All of the effective ratios are as calculated on a weight to weight basis (w/w).

A restoration and protection serum composition of the present invention comprises a composition comprising bioactive modulators (including, but not limited to, aloe barbadensis leaf juice, tocopheryl acetate, hydrolyzed rice protein, and glycine soya protein), Vitamin C, and a dermatologically-acceptable vehicle. An exfoliating facial mask composition of the present invention comprises bioactive modulators, skin cell exfoliating compounds, including but not limited to jojoba beads or spheres, kaolin, and a dermatologically-acceptable vehicle. A regenerating eye cream composition of the present invention comprises bioactive modulators, Vitamin K, and a dermatologically-acceptable vehicle. A skin moisturizer of the present invention comprises bioactive modulators and zinc oxide. The compositions taught herein are used in the methods and systems for treatment of skin.

Alternatively, effective amounts for the active ingredients, by weight percent, comprise differing amounts for differing compositions. For example, aloe barbadensis leaf juice (i.e., extract) may comprise a range of from about 0.05 weight percent to about 2.0 weight percent in an exfoliating facial mask, a regenerating eye cream, or a skin moisturizer. Alternatively, aloe barbadensis leaf juice may comprise a range of from about 0.1 weight percent to about 1.0 weight percent in an exfoliating facial mask, a regenerating eye cream, or a skin moisturizer. In a restoration and protection serum, the aloe barbadensis leaf juice may comprise a range of from about 85.0 weight percent to about 95.0 weight percent. Alternatively, a restoration and protection serum may comprise a range of aloe barbadensis leaf juice from about 88.0 weight percent to about 92.0 weight percent.

Vitamin E (i.e., tocopheryl acetate) may comprise a range of about 0.1 weight percent to about 2.0 weight percent in an exfoliating facial mask, a regenerating eye cream, a skin moisturizer, or a restoration and protection serum. Alternatively, Vitamin E may comprise a range of from about 0.5 weight percent to about 1.0 weight percent in an exfoliating facial mask, a regenerating eye cream, or a skin moisturizer.

Colhibin (hydrolyzed rice peptides) may comprise a range of about 0.2 weight percent to about 2.5 weight percent in an exfoliating facial mask, a regenerating eye cream, a skin moisturizer, or a restoration and protection serum. Alternatively, colhibin may comprise a range of from about 0.5 weight percent to about 1.5 weight percent in an exfoliating facial mask, a regenerating eye cream, a skin moisturizer, or a restoration and protection serum.

Elhibin (Soybean protein) may comprise a range of about 0.2 weight percent to about 2.5 weight percent in an exfoliating facial mask, a regenerating eye cream, a skin moisturizer, or a restoration and protection serum. Alternatively, elhibin may comprise a range of from about 0.5 weight percent to about 1.5 weight percent in an exfoliating facial mask, a regenerating eye cream, a skin moisturizer, or a restoration and protection serum.

The compositions of the present invention comprise a dermatologically-acceptable vehicle. This substance may act as a diluent, dispersant or carrier for the active ingredients. The vehicle may comprise materials commonly employed in skin care products, including but not limited to water, a buffered aqueous solution, liquid or solid emollients, silicone oils, emulsifiers, solvents, humectants, thickeners, powders, propellants and the like. The vehicle may constitute from approximately 5 percent to 99.9 percent by volume of the skin care composition, but may alternatively constitute from approximately 50 percent to 95 percent by volume of the skin care composition. In the absence of the other potential cosmetic or manufacturing adjuncts, the dermatologically acceptable vehicle will constitute the balance of the composition.

In addition to the active ingredients, the skin care compositions of the present invention may optionally contain various cosmetic or manufacturing adjuncts. For example, sunscreens, skin-lightening or skin-tanning agents may also be included. The vehicle may also further include adjuncts such as antioxidants, perfumes, opacifiers, preservatives, colorants and buffers, as necessary or desirable to enhance the efficacy, storage, utility, or marketability of the skin care composition. In some embodiments, the addition of perfumes or other masking agents to the skin care composition is desirable or necessary to reduce or block the odors associated with the presence of the active ingredients.

Exemplary oils which may be used according to this invention include, but are not limited to, mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate, polysorbate 60, polyethylene glycol-6 (PEG)-6))/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5 weight percent to about 50 weight percent. Alternatively, the levels of such emollients may range from about 5 weight percent to about 30 weight percent of the total composition. Emollients may be classified under such general chemical categories as esters, fatty acids and alcohols, polyols and hydrocarbons.

Esters may be mono- or di-esters. Examples of fatty di-esters include dibutyl adipate, diethyl sebacate, diisopropyl dimerate, and dioctyl succinate. Examples of branched chain fatty esters include 2-ethyl-hexyl myristate, isopropyl stearate and isostearyl palmitate. Examples of tribasic acid esters include triisopropyl trilinoleate and trilauryl citrate. Examples of straight chain fatty esters include lauryl palmitate, myristyl lactate, oleyl eurcate and stearyl oleate. Further examples of esters include coco-caprylate/caprate (a blend of coco-caprylate and coco-caprate), propylene glycol myristyl ether acetate, diisopropyl adipate and cetyl octanoate.

Examples of suitable fatty alcohols and acids include those compounds having from 10 to 20 carbon atoms. For example, such compounds such as cetyl, myristyl, palmitic, and stearyl alcohols and acids may be used.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol may be employed as penetration enhancers.

Hydrocarbons which may serve as emollients are those having hydrocarbon chains from about 12 to about 30 carbon atoms. Examples include mineral oil, petroleum jelly, squalene and isoparaffins.

Another category of functional ingredients within the cosmetic compositions of the present invention are thickeners. A thickener will usually be present in amounts anywhere from 0.1 weight percent to 20 weight percent; alternatively from about 0.5 weight percent to 10 weight percent of the composition. Exemplary thickeners are cross-linked polyacrylate materials available under the trademark Carbopol®. Gums may be employed such as xanthan, carrageenan, gelatin, karaya, pectin and locust beans gum. Under certain circumstances the thickening function may be accomplished by a material also serving as a silicone or emollient. For instance, silicone gums in excess of about 10 centistokes and esters such as glycerol stearate have dual functionality.

Powders may be incorporated into the cosmetic composition of the invention. These powders include, but are not limited to, chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range from about 0.001 weight percent up to about 20 weight percent of the composition.

Suitable vehicles include, but are not limited to, water, alcohols, oils, and gels chosen for their ability to dissolve or disperse the active ingredients at concentrations most suitable for therapeutic treatment and known to those of ordinary skill in the art. A preferred vehicle for the present invention includes a composition conducive to topical application, e.g., a carrier that will form a film or layer on the skin or one that aids in transcutaneous delivery and penetration of the active ingredients into lipid layers of the dermis. In addition, any of several forms of Vitamin C may be added to improve the efficacy of the above combinations of ingredients. The forms of Vitamin C include ascorbic acid, chitosan ascorbate, or magnesium ascorbyl phosphate.

The present invention comprises methods of using the compositions of the present invention for the treatment of skin and for the prevention of skin aging. In use, a small quantity of the composition, for example from about 1 milliliter (ml) to about 100 ml, is applied to exposed areas of the skin, from a suitable container or applicator and, if necessary, it is then spread over or rubbed into the skin using the hand or fingers or a suitable device. The product may be specifically formulated for use as a hand or as a facial treatment.

The methods of the present invention comprise treating and regulating mammalian skin conditions. Such regulation of skin conditions may include prophylactic and therapeutic regulation. For example, such regulating methods are directed to thickening keratinous tissue (e.g., building the epidermis or dermis layers of the skin and preventing or retarding atrophy of mammalian skin, preventing or retarding the appearance of spider vessels or red blotchiness on mammalian skin, preventing or retarding the appearance of dark circles under the eye of a mammal, preventing or retarding sallowness of mammalian skin, preventing or retarding sagging of mammalian skin, softening or smoothing lips, hair and nails of a mammal, preventing or relieving itch of mammalian skin, regulating skin texture, such as wrinkles and fine lines, and improving skin color as it relates to redness and freckles).

Methods may comprise topically applying to the keratinous tissue a safe and effective amount of a composition of the present invention. The amount of the composition which is applied, the frequency of application and the period of use will vary widely depending upon the effective amount of the active agents desired to be applied and the level of regulation desired, for example, the need for correction of damage in light of the level of keratinous tissue damage present or expected to occur.

Methods of use comprise methods of administration wherein the composition is chronically applied to the skin. “Chronic topical application” typically means continued topical application of the composition over an extended period during the subject's lifetime, for a period of at least about one week, for a period of at least about one month, for at least about three months, for at least about six months, or for at least about one year. While benefits are obtainable after various maximum periods of use, such as five, ten or twenty years, it is thought that chronic application may continue throughout the subject's lifetime. Typically applications may occur at least once per day over such extended periods, however, application rates can vary from about once per week up to about three times per day or more.

A wide range of quantities of the compositions of the present invention may be employed to provide a benefit to skin feel and appearance. Quantities of the present compositions which are typically applied per application are, in milligrams (mg) of composition per square centimeter (cm²) of skin, from about 0.1 mg/cm² to about 10 mg/cm². For example, one application amount is about 1 mg/cm² to about 2 mg/cm².

Systems for treating or regulating skin or keratinous tissue conditions are practiced by applying one or more compositions, in a particular order, in the form of a skin lotion, cream, gel, foam, ointment, paste, emulsion, spray, conditioner, tonic, cosmetic, lipstick, foundation, after-shave, or the like. The system for treating skin may include one or more compositions for preparing the skin. The skin preparation composition may be, for example, a cleanser, a mask, or a peel composition for some esthetic, prophylactic, therapeutic, or other benefit. For example, the skin preparation composition may be a cleanser or a peel composition designed to stay on the skin or other keratin structure for no longer than three minutes. Alternatively, the system for treating skin may include compositions left on the skin or other keratin structure, such as a “leave-on” composition. The system for treating skin may further include applying a skin care effective composition including at least one bioactive modulator and a dermatologically acceptable vehicle. The bioactive modulator may be, for example, aloe barbadensis, tocopheryl acetate, hydrolyzed rice protein, or glycine soya protein. The bioactive modulator may be incorporated in a skin toning composition, a moisturizer, or an eye cream. After applying the composition to the skin, it may be left on the skin for a period of at least about 15 minutes, at least about 30 minutes, at least about 1 hour, at least several hours, or up to 12 hours. Any part of the external portion of the face, hair, and/or hands, feet or skin can be treated, e.g., face, lips, under-eye area, eyelids, scalp, neck, torso, arms, hands, legs, feet, fingernails, toenails, scalp hair, eyelashes, eyebrows, etc. The composition can be applied with the fingers or with an implement or device, including, but not limited to, pad, cotton ball, applicator pen, or spray applicator.

In addition, the systems for treating skin may include a restoration and protection serum. The restoration and protection serum may be applied, for example, with makeup or incorporated in makeup.

Another approach to ensure a continuous exposure of the skin to at least a minimum level of the active agents is to apply the active agents by use of a patch applied to the intended skin area. Such an approach is particularly useful for problem skin areas needing more intensive treatment, such as facial “crows feet” area, frown lines, under eye area, and the like. The patch can be occlusive, semi-occlusive or non-occlusive and can be adhesive or non-adhesive. The composition can be contained within the patch or be applied to the skin prior to application of the patch. The patch can also include additional actives such as chemical initiators for exothermic reactions such as those described in U.S. Pat. Nos. 5,821,250, 5,981,547, and 5,972,957 to Wu, et al. The patch may be left on the skin for a period of at least about 5 minutes, for at least about 15 minutes, for at least about 30 minutes, for at least about 1 hour, or at night as a form of night therapy.

In one aspect, a method of using the instant invention may be enhanced by and may include preparation of the skin prior to carrying out method steps. Preparation of the skin may include cleaning the skin with a cleansing agent to clear the skin of excess oil and debris (e.g., dirt and excess dead cells). Preparation of the skin may further include applying a composition, such as a facial cleanser or a peel composition, which peels one or more outer layers of skin to clear excess oil and debris. Preparation of the skin may further include toning the skin with a toning agent to balance the pH and optimize the barrier property of the skin for receiving the bioactive modulator blend.

By way of example, a typical user of the invention may first wash his or her skin using a cleansing agent. See Table 1 for an exemplary formulation for a facial cleansing agent. See Table 2 for an exemplary formulation for a peel composition. The cleansing agent will remove excess oil and debris from the skin to allow easier access for the biomodulator blend of the present invention. Next, a typical user may further prepare his or her skin using a skin toner. See Table 3 for an exemplary formulation for a skin toner. The skin toner may further prepare the skin by balancing the pH of the skin and adjusting the moisture content of the skin (thus adjusting the barrier properties of the skin).

Following these steps, the method of the invention may be used to treat and improve the appearance of the skin. For example, a typical user may choose to exfoliate his or her skin using the exfoliant of the present invention (See Table 5) followed by treatment with the skin moisturizer of the present invention (See Table 4). Next, a typical user may further treat his or her skin using the regenerating eye cream of the present invention (See Table 6) or the restoration and protection serum of the present invention (See Table 7). All or part of the method of the invention may be used, depending on the skin needs of a typical user.

In another embodiment, methods of the present invention comprise making the compositions taught herein. To prepare the skin care composition of the present invention, a variety of techniques may be employed. For example, the bioactive modulators may be generally incorporated into the dermatologically-acceptable vehicle in the manner that is usual for the preparation of skin care products. Thus, the bioactive modulators may first be dissolved or dispersed in a portion of the water or another solvent or liquid to be incorporated into the dermatologically-acceptable vehicle. Compositions for use in this manufacturing approach may be oil-in-water, water-in-oil, or water-in-oil-in-water emulsions. Other forms such as lotions, creams, or other delivery vehicles known to those skilled in the art are contemplated by the present invention.

The foregoing description includes the best presently contemplated mode of carrying out the present invention. This description is made for the purpose of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

All terms used herein are considered to be interpreted in their normally accepted usage by those skilled in the art. Patent and patent applications or references cited herein are each incorporated by reference in its entirety.

### EXAMPLES

### Example 1

### Exemplary Formulations of the Present Invention May Comprise the Following

TABLE 1
Facial Cleanser.

| Ingredient | Percentage |
|---|---|
| Water | 55.0–60.0 |
| Disodium EDTA | 0.05 |
| PEG-80 Sorbitan Laurate | 30.0–35.0 |
| Phenonxyethanol, Methylparaben, Butylparaben, | 0.5 |
| Ethylparaben, Propylparaben | |
| Panthenol | 0.5 |
| Aloe Barbadensis Leaf Extract | 0.5 |
| Glycereth-26 | 2.0–5.0 |
| Dimethicone PEG-7 Isostearate | 0.0–0.75 |
| Bisabolol | 0.5–0.75 |
| Polysorbate 20 | 1.0–1.5 |
| PEG-40 Castor Oil | 0.0–0.5 |
| Hedera Helix (Ivy) Extract, Sambucus Nigra | 2.5 |
| (Elder) Flower extract, Arnica Montana Flower | |
| Extract, Althea Officinalis Root Extract, | |
| Cucumis Sativus (Cucumber) Extract Anacyclus | |
| Pyrethrum Root (Pellitory) Extract* | |
| Tilia Cordata (Linden) Flower Extract, Calendula | 2.5 |
| Officinalis Flower Extract, Centaurea Cyanus | |
| (Cornflower) Flower Extract, Chamomilla | |
| Recutita Flower Extract, Anthemis Nobilis | |
| (Roman Chamomile) Flower Extract, Hypericum | |
| Perforatum Extract** | |
| Salvia Officinalis (Sage) Leaf Extract, Panax | 2.5 |
| Ginseng Root Extract, Equisetum Hiemale | |
| (Horsetail) extract, Nasturtium Officinale | |
| Extract, Cymbopogon Schoenanthus | |
| (Lemongrass) Extract*** | |
| Salicylic acid*** | 0.5 |

*Used in Normal skin formulations
**Used in dry skin formulations
***Used in oily skin formulations
Numerical values represent percentages based on a maximum of 100%.

TABLE 2
Peel Composition

| Ingredient | Percentage |
|---|---|
| Water | 40.0–50.0 |
| SD Alcohol 40 | 25.0–35.0 |
| Lactic acid | 8.5–10.5 |
| Mandelic acid | 5.0–7.0 |
| Malic acid | 1.0–5.0 |
| Tartaric acid | 1.0–5.0 |
| Salicylic acid | 0.5–5.0 |
| Hydroxypropyl Methyl Cellulose | 0.5–5.0 |
| Disodium EDTA | 0.05–0.5 |

TABLE 3
Skin Toner.

| Ingredient | Percentage |
|---|---|
| Water | 48.0–78.0 |
| SD Alcohol 40 | 10.0–40.0 |
| Glycerin, Hamamelis Virginiana (Witch Hazel) | 5.0 |
| Extract(* and **) | |
| Acetamide MEA*** | 5.0 |
| Panthenol | 0.5 |
| Aloe Barbadensis Leaf Extract | 0.1 |
| Glycerin(* and **) | 5.0 |
| Salicylic acid*** | 0.5 |
| Phenonxyethanol, Methylparaben, Butylparaben, | 0.5 |
| Ethylparaben, Propylparaben(* and **) | |
| Dimethicone PEG-7 Cocoate(* and **) | 1.0 |
| Nonoxynol-9*** | 0.5 |

*Used in Normal skin formulations
**Used in dry skin formulations
***Used in oily skin formulations
Numerical values represent percentages based on a maximum of 100%.

TABLE 4
Skin Moisturizer. Numerical values represent percentages based on a
maximum of 100%.

| | |
|---|---|
| Ingredient | Percentage |

PART A

| | |
|---|---|
| Water | 61.0–64.0 |
| Glycerin | 5.0 |
| Disodium EDTA | 0.05 |
| Methylparaben | 0.3 |

PART B

| | |
|---|---|
| Cyclopentasiloxane | 15.0 |
| Dimethicone, Cyclopentasiloxane | 0.5–3.0 |
| Isostearyl linoleate | 0.5–2.0 |
| Caprylic/capric triglyceride | 0.5–2.0 |
| Ceteareth-20 | 2.0–3.0 |
| Propylparaben | 0.2 |
| Squalene(* and **) | 2.0–3.0 |
| Cetyl Alcohol | 0.75 |
| Stearyl Alcohol | 0.5 |
| Zinc Oxide | 5.0 |

PART C

| | |
|---|---|
| Hydrolyzed Rice Protein | 0.2–2.5 |
| Glycine Soja (Soybean) protein | 0.2–2.5 |
| Aloe Barbadensis Leaf Extract | 0.05–2.0 |
| DMDM Hydantoin | 0.5 |
| Tocopheryl Acetate | 0.1–2.0 |
| Allantoin(* and **) | 0.5–0.75 |
| Argania Spinosa Kernel Oil, Serenoa Serrulata | 5.0 |
| Fruit Extract, Sesamum Indicum (Sesame) Seed | |
| Extract(***) | |

PART D

| | |
|---|---|
| Water | 45.0–51.0 |
| Acrylates/Acrylamide Copolymer 26–28% | 0.75 |
| NF Grade Light Mineral Oil 22–24% | |
| Polysorbate 85 1–3% | |

*Used in Normal skin formulations
**Used in dry skin formulations
***Used in oily skin formulations
Numerical values represent percentages based on a maximum of 100%.

TABLE 5
Exfoliating facial mask.

| | |
|---|---|
| Ingredient | Percentage |

PART A

| | |
|---|---|
| Water | 30.25 |
| Kaolin | 20.0 |
| Magnesium Aluminum Silicate | 0.75 |

PART B

| | |
|---|---|
| Water | 10.0 |
| Glycerin | 5.0 |
| Disodium EDTA | 0.05 |
| Sodium methyl cocoyl taurate | 5.0 |

PART C

| | |
|---|---|
| Isostearyl linoleate | 2.0 |
| Caprylic/capric triglyceride | 3.0 |
| Ceteareth-20 | 3.5 |
| Dimethicone | 0.25 |
| Glyceryl Stearate, PEG-100 Stearate | 4.8 |
| Squalene | 1.0 |
| Bisabolol | 1.0 |
| Tocopheryl Acetate | 0.1–2.0 |

PART D

| | |
|---|---|
| Hydrolyzed Rice Protein | 0.2–2.5 |
| Glycine Soja (Soybean) protein | 0.2–2.5 |
| Panthenol | 0.1 |
| Aloe Barbadensis Leaf Extract | 0.05–2.0 |
| Phenonxyethanol, Methylparaben, Butylparaben, | 1.2 |
| Ethylparaben, Propylparaben | |
| Hydrogenated Jojoba Oil | 10.0 |

*Used in Normal skin formulations
**Used in dry skin formulations
***Used in oily skin formulations
Numerical values represent percentages based on a maximum of 100%.

TABLE 6
Regenerating eye cream.
PART A

| | |
|---|---|
| Water | 69.5 |
| Acrylates/C10–30 Alkyl Acrylate Crosspolymer | 0.15 |
| Glycerin | 5.0 |
| Disodium EDTA | 0.05 |

PART B

| | |
|---|---|
| Octoxynol-9 | 7.5 |
| Isostearyl linoleate | 5.0 |
| Caprylic/capric triglyceride | 2.5 |
| Dimethicone, Trimethylsiloxysilicate | 0.25 |
| Tocopheryl Acetate | 0.1–2.0 |
| Phytonadione (Vitamin K1) | 0.1 |
| Glyceryl Stearate, | 2.0 |
| PEG-100 Stearate | |
| Cetyl alcohol | 0.75 |
| Stearyl alcohol | 0.5 |

PART C

| | |
|---|---|
| Water | 0.5 |
| Triethanolamine | 0.2 |

PART D

| | |
|---|---|
| Hydrolyzed Rice Protein | 0.2–2.5 |
| Glycine Soja (Soybean) protein) | 0.2–2.5 |
| Aloe Barbadensis Leaf Extract | 0.05–2.0 |
| Magnesium Ascorbyl Phosphate | 0.2 |
| Phenonxyethanol, Methylparaben, Butylparaben, | 1.0 |
| Ethylparaben, Propylparaben | |

PART E

| | |
|---|---|
| Water | 45.0–51.0 |
| Acrylates/Acrylamide Copolymer 26–28%, | 1.5 |
| NF Grade Light Mineral Oil 22–24%, | |
| Polysorbate 85 1–3% | |

TABLE 7
Restoration and protection serum.
Part A

| | |
|---|---|
| Aloe Barbadensis Leaf Extract | 85.0–95.0 |
| Glycerin | 3.0 |
| Disodium EDTA | 0.05 |
| Superoxide Dismutase | 0.1 |
| Hydrolyzed Rice Protein | 0.2–2.5 |
| Glycine Soja (Soybean) protein) | 0.2–2.5 |
| DMDM-Hydantoin | 0.25 |
| Tocopheryl Acetate | 0.1–2.0 |
| Polysorbate 20 | 1.0 |

Part B

| | |
|---|---|
| Chitosan Ascorbate | 100.0 |

Mix the two Parts for application to the skin.

### Example 2

### Methods for Making Exemplary Formulations

In another aspect, the invention is a method for making the skin care system. With respect to the facial cleanser, the peel composition, and the skin toner, (Tables 1, 2, and 3, respectively), the method includes the steps of combining the ingredients in the order listed, mixing until clear between additions. The method may be performed at room temperature.

Regarding the skin moisturizer (Table 4), a method includes the steps of combining the Part A ingredients into a main kettle equipped with a sweep mixer, agitating the ingredients, and heating the ingredients to a range of from about 70° C. (Centigrade) to about 75° C. A method further includes the steps of combining Part B ingredients in a side kettle equipped with a homogenizer and propeller mixer, heating the Part B ingredients to a range of from about 70° C. to about 75° C. while mixing, and homogenizing for about ten minutes after the zinc oxide is added. The method may next include the steps of combining Part A with Part B with moderate mixing, maintaining a temperature range of about 70° C. to about 75° C. for about thirty minutes and thereafter cooling the mixture to a range of from about 40° C. to about 45° C. Following this, the method may include the steps of adding the ingredients of Part C individually, mixing until uniform between additions. Finally, the method may include the step of adding Part D and mixing for at least about ten minutes.

A method of the invention for the exfoliating facial mask of the skin care system (Table 5) includes the steps of combining the ingredients of Parts A, B, C, and D in order with moderate agitation and until clear between additions.

A method of the invention for the regenerating eye cream (Table 6) of the skin care system includes the steps of charging a main sweep kettle with Part A water, followed by sprinkling the Acrylates/C10-30 Alkyl Acrylate Crosspolymer on the surface with no agitation, allowing the Acrylates/C10-30 Alkyl Acrylate Crosspolymer to self-wet. Next, the steps of agitating, adding the remaining Part A ingredients, and heating the Part A ingredients to a range of from about 70° C. to about 75° C. The method may further call for the steps of combining Part B items in a side kettle equipped with a mixer and heating to a range of from about 70° C. to about 75° C. Next, when both Parts A and B are uniform and at temperature, the method may include combining Parts A and B with moderate mixing. The method may also call for the steps of pre-mixing Part C ingredients, adding the Part C mixture to the mixture of Parts A and B, and then mixing the combination of Parts A, B, and C at a temperature range of about 70° C. to about 75° C. for about 30 minutes, followed by the step of cooling the mixture to a range of from about 40° C. to about 45° C. Next, the method of the invention includes adding Part D ingredients individually, mixing until uniform between additions, followed by the step of cooling the mixture to a range of about 30° C. to about 35° C. Finally, the method of the invention for the regenerating eye cream may include the steps of adding Part E and mixing for about ten minutes.

A method of the invention for the restoration and protection serum (Table 7) of the skin care system includes the steps of combining the ingredients in the order listed, mixing between additions until uniform.

In another aspect, all or part of the invention may be used to decrease the inflammatory response, improve moisture levels, increase cellular antioxidant capabilities, increase collagen production, and decrease MMP activity to various degrees. For example, the facial cleanser, the skin toner, and the skin moisturizer may be used on a daily basis to provide and maintain the above-mentioned benefits. Following this treatment, the skin care system of the present invention may also provide for treating skin imperfections using the exfoliating face mask, the regenerating eye cream, and the restoration and protection serum.

1. A skin care effective composition, comprising,
at least one bioactive modulator and a dermatologically acceptable vehicle, wherein the at least one bioactive modulator is aloe barbadensis, tocopheryl acetate, hydrolyzed rice protein, or glycine soya protein.

2. The composition of claim 1, wherein the dermatologically acceptable vehicle comprises water, a buffered aqueous solution, liquid or solid emollients, oils, emulsifiers, solvents, humectants, thickeners, powders, or propellants.

3. The composition of claim 2, wherein oils comprises mineral oils, plant oils, animal oils, synthetic oils, silicone oils, and perfluoropolyethers.

4. The composition of claim 2, wherein emulsifiers comprises glyceryl stearate, polysorbate 60, and a polyethylene glycol-6 (PEG)-6/PEG-32/glycol stearate mixture.

5. The composition of claim 2, wherein thickeners comprises between about 0.1 percent to about 20 percent, by weight, of cross-linked polyacrylates, xanthan gum, carrageenan, gelatin, karaya, pectin, locust bean gum, silicone gum, and glycerol stearate.

6. The composition of claim 2, wherein powders comprises chalk, talc, kaolin, starch, smectite clays, chemically modified magnesium aluminum silicate, organically modified montmorillonite clay, hydrated aluminum silicate, fumed silica, aluminum starch octenyl succinate, and mixtures thereof.

7. The composition of claim 1, further comprising sunscreens, skin-lightening agents, skin-tanning agents, antioxidants, perfumes, opacifiers, preservatives, colorants, or buffers.

8. The composition of claim 1, wherein the ratio of tocopheryl acetate to the dermatologically acceptable vehicle is between about 1:675 to about 1:500 as calculated on a weight to weight basis.

9. The composition of claim 1, wherein the ratio of hydrolyzed rice protein to the dermatologically acceptable vehicle is between about 1:200 to about 1:50 as calculated on a weight to weight basis.

10. The composition of claim 1, wherein the ratio of glycine soya protein to the dermatologically acceptable vehicle is between about 1:200 to about 1:50 as calculated on a weight to weight basis.

11. A method for treatment and maintenance of skin, comprising,
administering an effective amount of a skin care effective composition, comprising,
at least one bioactive modulator and a dermatologically acceptable vehicle, wherein the at least one bioactive modulator is aloe barbadensis, tocopheryl acetate, hydrolyzed rice protein, or glycine soya protein.

12. The method of claim 11, wherein administering an effective amount of the skin care effective composition comprises administering between about 0.1 milligrams per square centimeter of skin to about 10 milligrams per square centimeter of skin.

13. The method of claim 11, further comprising preparation of the mammalian skin with a cleansing composition prior to administering the skin care effective composition.

14. The method of claim 11, further comprising preparation of the mammalian skin with a skin toning composition prior to administering the skin care effective composition.

15. The method of claim 11, further comprising preparation of the mammalian skin on a periodic basis by administering a mask composition.

16. The method of claim 11 further comprising preparation of the mammalian skin on a periodic basis by administering a peel composition.

17. The method of claim 11, wherein administering comprises applying an effective amount of one or more compositions that decrease the skin inflammatory response, provides antioxidant capability, improves moisture levels or decreases MMP activity.

18. A system for skin treatment, comprising,
preparing skin using a cleansing composition to remove excess oil, dirt, and debris, and
applying a skin care effective composition comprising at least one bioactive modulator and a dermatologically acceptable vehicle, wherein the at least one bioactive modulator is aloe barbadensis, tocopheryl acetate, hydrolyzed rice protein, or glycine soya protein.

19. The system of claim 18, further comprising,
applying a skin toning composition, a moisturizer, or an eye cream following the step of preparing the skin.

20. The system of claim 18, further comprising, preparing the skin using a peel composition and wherein the skin care effective composition is a mask.
